(19) United States
(12) Patent Application Publication
(10) Pub. No.: US 2022/0022756 A1
(43) Pub. Date: Jan. 27, 2022

(54) Providing Image Units for Vital Sign Monitoring

(71) Applicant: Teknologian tutkimuskeskus VTT Oy, Espoo (FI)

(72) Inventors: Tero Kiuru, Espoo (FI); Mikko Metso, Espoo (FI); Mervi Hirvonen, Espoo (FI); Tauno Vähä-Heikkilä, Espoo (FI)

(21) Appl. No.: 17/312,096

(22) PCT Filed: Dec. 3, 2019
(86) PCT No.: PCT/FI2019/050865

§ 371 Date: Jun. 9, 2021

(30) Foreign Application Priority Data
Dec. 18, 2018 (FI) 20186107

(51) Int. Cl. A61B 5/01; A61B 5/0507; A61B 5/024; A61B 5/08; G01S 13/89; A61B 5/00

(52) U.S. Cl. CPC A61B 5/015; A61B 5/0507; A61B 5/024; A61B 5/0013; G01S 13/89; A61B 5/0008; A61B 5/0816

(57) ABSTRACT

According to an example aspect of the present invention, there is provided image units for vital sign monitoring by a multichannel radar scanning a field-of-view and a microwave imaging radiometer sounding the field-of-view. Presence of moving targets within the field-of-view of the radar is determined on the basis of phase and/or amplitude changes of the image units between scans. Thermal information or information determined based on the thermal information obtained by the sounding is combined with the image units of the moving targets. In this way vital signs may be monitored based on movement and thermal information of the image units.

### FIELD

The present invention relates providing image units for vital sign monitoring by a multichannel radar and microwave imaging radiometer.

### BACKGROUND

Error sources in remote vital sign monitoring can hinder or even prevent to accurately determine the vital signs such as breathing rate and heart rate. The error sources can be specific to technique used for remote vital sign monitoring.

Doppler and/or UWB impulse radar techniques are used for remote vital sign monitoring. These techniques provide measuring breathing of a person. However, these techniques operate in low microwave frequencies and therefore, their angular resolution is limited, particularly close to the radar such as indoors in living facilities. Improvement of the angular resolution by enlarging the antenna systems introduces limitations to use of the radar in indoor installations.

Compensating error sources of Doppler and/or UWB impulse radar techniques is not sufficient for accurate monitoring of vital signs if other techniques are used in additionally for vital sign monitoring.

### SUMMARY OF THE INVENTION

The invention is defined by the features of the independent claims. Some specific embodiments are defined in the dependent claims.

According to a first aspect of the present invention, there is provided a method of providing image units for vital sign monitoring, comprising:
scanning, by a multichannel radar or at least one processing unit connected to the radar, a field-of-view within a frequency range from 1 to 1000 GHz, for example between 1 to 30 GHz, 10 to 30 GHz, 30 to 300 GHz or 300 to 1000 GHz, using a plurality of radar channels of the radar;
generating, by the radar or the processing unit connected to the radar, image units for a radar image on the basis of results of the scanning, wherein the image units comprise at least amplitude and phase information;
determining, by the radar or the processing unit connected to the radar, a presence of moving targets within the field-of-view of the radar on the basis of phase and/or amplitude changes of the image units between scans;
sounding the field-of-view by a microwave imaging radiometer for obtaining a thermal information associated with the field-of-view;
combining the thermal information obtained by the microwave imaging radiometer with the image units of the moving targets.

According to a second aspect of the present invention, there is provided an arrangement comprising:
multichannel radar, and
microwave imaging radiometer, wherein arrangement is configured to cause:
   scanning, by the multichannel radar or at least one processing unit connected to the radar, a field-of-view within a frequency range from 1 to 1000 GHz, for example between 1 to 30 GHz, 10 to 30 GHz, 30 to 300 GHz or 300 to 1000 GHz, using a plurality of radar channels of the radar;
   generating, by the radar or the processing unit connected to the radar, image units for a radar image on the basis of results of the scanning, wherein the image units comprise at least amplitude and phase information;
   determining, by the radar or the processing unit connected to the radar, a presence of moving targets within the field-of-view of the radar on the basis of phase and/or amplitude changes of the image units between scans;
   sounding the field-of-view by the microwave imaging radiometer for obtaining a thermal information associated with the field-of-view;
   combining the thermal information obtained by the microwave imaging radiometer with the image units of the moving targets.

According to a third aspect there is provided a non-transitory computer readable medium having stored thereon a set of computer readable instructions that, when executed by at least one processor, cause an arrangement to at least:
scanning, by a multichannel radar or at least one processing unit connected to the radar, a field-of-view within a frequency range from 1 to 1000 GHz, for example between 1 to 30 GHz, 10 to 30 GHz, 30 to 300 GHz or 300 to 1000 GHz, using a plurality of radar channels of the radar;
generating, by the radar or the processing unit connected to the radar, image units for a radar image on the basis of results of the scanning, wherein the image units comprise at least amplitude and phase information;
determining, by the radar or the processing unit connected to the radar, a presence of moving targets within the field-of-view of the radar on the basis of phase and/or amplitude changes of the image units between scans;
sounding the field-of-view by a microwave imaging radiometer for obtaining a thermal information associated with the field-of-view;
combining the thermal information obtained by the microwave imaging radiometer with the image units of the moving targets.

According to a fourth aspect there is provided a computer program comprising instructions for causing an arrangement to perform at least the following:
scanning, by a multichannel radar or at least one processing unit connected to the radar, a field-of-view within a frequency range from 1 to 1000 GHz, for example between 1 to 30 GHz, 10 to 30 GHz, 30 to 300 GHz or 300 to 1000 GHz, using a plurality of radar channels of the radar;
generating, by the radar or the processing unit connected to the radar, image units for a radar image on the basis of results of the scanning, wherein the image units comprise at least amplitude and phase information;
determining, by the radar or the processing unit connected to the radar, a presence of moving targets within the field-of-view of the radar on the basis of phase and/or amplitude changes of the image units between scans;
sounding the field-of-view by a microwave imaging radiometer for obtaining a thermal information associated with the field-of-view;
combining the thermal information obtained by the microwave imaging radiometer with the image units of the moving targets.

According to a fifth aspect of the present invention, there is provided a method for monitoring living facilities by a multichannel radar or an arrangement comprising a multichannel radar:
scanning, by a multichannel radar or at least one processing unit connected to the radar, a field-of-view within a frequency range from 1 to 1000 GHz, for example between 1 to 30 GHz, 10 to 30 GHz, 30 to 300 GHz or 300 to 1000 GHz, using a plurality of radar channels of the radar;
generating, by the radar or the processing unit connected to the radar, a radar image on the basis of results of the scanning, wherein the radar image comprises image units comprising at least amplitude and phase information;
identifying from the radar image, by the radar or the processing unit connected to the radar, separate sets of image units on the basis of the amplitude and/or phase information of the image units; and
determining, by the radar or the processing unit connected to the radar, a presence of moving targets within the field-of-view of the radar on the basis of phase and/or amplitude changes of the image units between scans.

According to a sixth aspect of the present invention, there is provided a multichannel radar or an arrangement comprising a multichannel radar for monitoring living facilities, comprising:
means for scanning a field-of-view within a frequency range from 1 to 1000 GHz, for example between 1 to 30 GHz, 10 to 30 GHz, 30 to 300 GHz or 300 to 1000 GHz, using a plurality of radar channels of the radar;
means for generating a radar image on the basis of results of the scanning, wherein the radar image comprises image units comprising at least amplitude and phase information;
means for identifying from the radar image separate sets of image units on the basis of the amplitude and/or phase information of the image units; and
means for determining a presence of moving targets within the field-of-view of the radar on the basis of phase changes of the image units between scans.

Further aspects of the present invention may comprise one or more of:
monitoring phases of image units of one or more moving targets and if the image units indicate large movements based on a phase change of the image units and/or a change of periodicity of the image units, the thermal information associated with the image units is compensated;
wherein the compensation is applied during a time period;
updating the image units with thermal information or information determined based on the thermal information obtained by at least one sounding performed after the time period has passed.
determining a temperature representative of a body temperature of at least one of the moving targets based on the thermal information obtained by the microwave imaging radiometer;
determining image units associated with a person in the field-of-view; determining the image units associated with the person to indicate a sign of life, a temperature, heart rate and/or a breathing rate;
a breathing and/or heart rate are determined based on periodical changes of phase together with relatively small changes of amplitude of an image unit;
determining a medical condition of a person based on a change pattern of the image units.
determining vital signs and/or a medical condition of a person based on the thermal information combined with the image units;
determining a number of the moving targets, on the basis of the number of the separate sets of the image units; and entering the radar to a power saving mode, when the number of moving targets is one or less, wherein the power saving mode comprises that the radar is controlled to scan the field-of-view using a reduced number of radar channels;
time interval between the scans is reduced, when the power saving mode is entered;
the radar is triggered to leave the power saving mode after a time interval has passed and/or on the basis of a trigger signal;
the power saving mode change patterns of the image units corresponding to micro movements such as at least one of heart rate and breathing are determined;
an artificial intelligence system and a user interface are connected to the radar or the processing unit to cause:
   i. —a) obtaining user input indicating a number of targets within the field-of-view;
   ii. —b) identifying separate sets of image units corresponding to targets from a generated radar image;
   iii. —c) determining a correspondence between separate sets of image units of the generated radar image and the number of targets within the field-of-view indicated by the user input; and
   iv. —d) re-configuring the artificial intelligence system, when the correspondence is not determined; and repeating phases a) to d) until the correspondence between separate sets of image units of radar images and the number of targets within the field-of-view indicated by the user input is obtained with sufficient certainty, for example 99% certainty.
the image units belonging to the separate sets are determined by grouping the image units on the basis of at least one of:
   i. —range of the image units;
   ii. —azimuth angle of the image units
   iii. —elevation angle of the image units; and
   iv. —phase and/or amplitude changes between of the image units between the scans.
the moving targets comprise a plurality of types, for example pets, humans, children and/or adults.
displaying at least one of the radar image, information indicating the number of moving targets, types of the moving targets, information indicating heart rate and information indicating breathing.

### BRIEF DESCRIPTION OF THE DRAWINGS

FIG. 1 illustrates an example of a multichannel radar in accordance with at least some embodiments of the present invention;

FIG. 2 illustrates an example of a method in accordance with at least some embodiments of the present invention;

FIG. 3 illustrates an example of a radar image in accordance with at least some embodiments of the present invention;

FIG. 4 illustrates an example of a radar image in accordance with at least some embodiments of the present invention;

FIG. 5 illustrates an example of a method for controlling a multichannel radar in accordance with at least some embodiments of the present invention;

FIG. 6 illustrates configuring an artificial intelligence system in accordance with at least some embodiments of the present invention; and

FIG. 7 illustrates an example of an arrangement in accordance with at least some embodiments of the present invention;

FIG. 8 illustrates an example of a method in accordance with at least some embodiments of the present invention;

FIG. 9 illustrates an example of a method in accordance with at least some embodiments of the present invention;

FIG. 10 illustrates an example of a method in accordance with at least some embodiments of the present invention;

FIG. 11 illustrates an example of antenna array layout of a microwave imaging radiometer in accordance with at least some embodiments of the present invention; and

FIG. 12 illustrates an example of a receiver for an antenna element of a microwave imaging radiometer in accordance with at least some embodiments of the present invention; and

FIG. 13 illustrates an example of a method for sounding a field-of-view by a microwave imaging radiometer in accordance with at least some embodiments of the present invention.

### EMBODIMENTS

In the present context a multichannel radar may refer to a Multiple Input Multiple Output (MIMO) radar comprising a system of multiple transmitting antennas and multiple receiving antennas, a Multiple Input Single Output (MISO) radar comprising a system of multiple transmitting antennas and a single receiving antenna or a Single Input Multiple Output (SIMO) radar comprising a system of a single transmitting antenna and multiple receiving antennas. The transmitting antennas may be configured to radiate a signal waveform in a region of the electromagnetic spectrum independently of the other transmitting antennas. Each receiving antenna can receive these signals, when the transmitted signals are reflected back from a target in a field-of-view of the radar. The transmitted waveforms are distinguishable from each other such that they may be separated, when they are received by the receiving antennas.

In the present context living facilities refers to buildings and premises or their parts such as rooms, used by people and/or pets. Examples of the living facilities comprise offices, homes, home care facilities, assisted living facilities, nursing homes and hospitals.

A radar channel is a combination of transmitting antenna and receiving antenna. A signal waveform transmitted by a multichannel radar comprising k transmitting antennas and n receiving antennas may be received via k x n radar channels. In an example, k=4 and n=8, whereby the number of radar channels becomes 32.

An active radar channel refers to a combination of transmit and receive antennas that are in use for transmitting and receiving operation.

A passive radar channel refers to a combination of transmit and receive antennas that are not in use for transmitting and receiving operation.

Scanning a field-of-view by multichannel radar refers to transmitting a signal waveform by transmitting antennas of the multichannel radar and receiving reflected copies of the transmitted signal waveform by receiving antennas of the multichannel radar. The scanning is performed by active radar channels. In this way results of the scanning comprising signal waveforms of all the active radar channels defined by the transmitting antennas and receiving antennas are obtained.

Monitoring living facilities is provided by a multichannel radar, by scanning a field-of-view using a plurality of transmit and receive channels of the radar. A radar image is generated based on results of the scanning. Separate sets of image units are identified from the radar image on the basis of amplitude and/or phase information of the image units. Presence of moving targets within the field-of-view is determined on the basis of phase and/or amplitude changes of the image units between scans. The movement of the targets is reflected in the amplitude and/or phase of the scans, whereby the targets may be determined as moving targets. In this way living facilities may be monitored without a live camera view from the living facilities. Since the monitoring is performed based on the radar image, the monitoring may be performed without compromising privacy of the people and/or the living facilities.

Monitoring living signs is provided by a multichannel radar scanning a field-of-view and a microwave imaging radiometer sounding the field-of-view. Presence of moving targets within the field-of-view of the radar is determined on the basis of phase and/or amplitude changes of the image units between scans. Thermal information or information determined based on the thermal information obtained by the sounding is combined with the image units of the moving targets. In this way vital signs may be monitored based on movement and thermal information of the image units, whereby the movement information may be used to adaptively process the thermal information.

A moving target may refer to a target, for example a pet or a person, or a part of the target, that is moving.

A micro movement may be a movement of a part of the target, for example a movement of the chest by respiration or a movement of the chest by heartbeat.

An image unit refers to a point in a radar image that may be controlled to be displayed on a user interface. The imaging unit may be an image element, for example a pixel, in digital imaging.

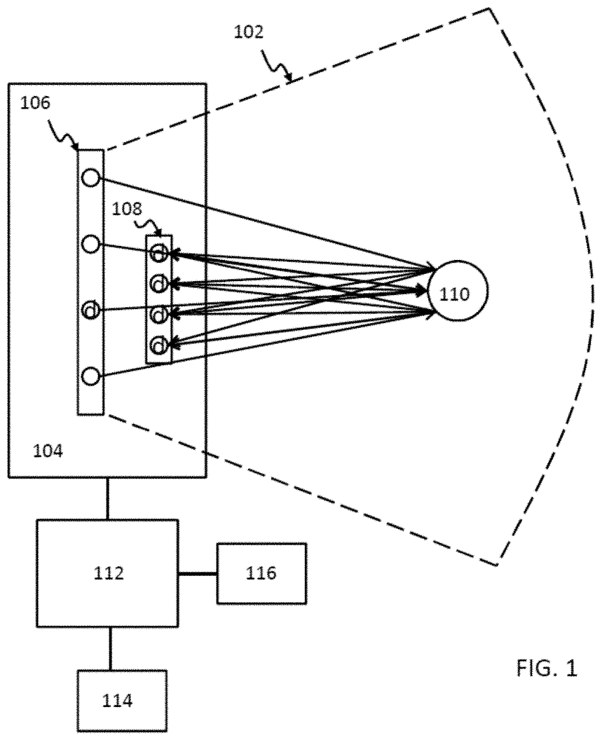

FIG. 1 illustrates an example of multichannel radar in accordance with at least some embodiments of the present invention. The multichannel radar 104 comprises a plurality transmitting antennas 106 and a plurality of receiving antennas 108 for scanning a field-of-view 102 of the radar for a presence of one or more targets 110 within the field-of-view by radar channels defined by combinations of the transmits and receive channels. The radar is configured to perform the scanning within a frequency range of 1 to 1000 GHz, for example between 1 to 30 GHz, 10 to 30 GHz, 30 to 300 GHz or 300 to 1000 GHz, whereby signal waveforms are transmitted by the transmitting antennas at a carrier frequency selected from the frequency range. The frequency range of 30 to 300 GHz or the higher frequency range of 300 to 1000 GHz may be preferred such that the radar may be configured to have dimensions suitable for indoor installations, while providing the radar to have a sufficient angular resolution. When a target is present within the field-of-view, transmitted signal waveforms are reflected from the target and received by the radar channels of the radar. Preferably, the scanning is performed using a number of radar channels that is sufficient for generating a radar image for determining presence of multiple moving targets within the living facilities. The number of radar channels affects resolution of the monitoring performed by the radar. For example 8 parallel radar channels provides a resolution of 14 degrees and 32 parallel radar channels provides a resolution of 3,5 degrees. In an example, 16 radar channels may be sufficient for monitoring a person that is walking. In an example the scanning may be performed between time intervals, whose duration may be determined based on the speed of movement of the moving targets. In a normal operation mode substantially all radar channels are active and used for scanning such that multiple moving targets may be identified from a radar image generated on the basis of results of the scanning. In a power saving operation mode a reduced number of radar channels are active, for example one active radar channel, and used for scanning such that a single moving target may be identified from a radar image generated on the basis of results of the scanning. In the power saving mode the time interval between scans may be reduced for example with respect to the scanning interval, such as the scanning interval in the normal operation mode, used before entering the power saving mode. A target identified from the radar image may be determined to be a moving target based on phase and/or amplitude changes of the image units of the radar images generated based on scans.

In an example the radar may comprise 4 transmitting antennas and 8 receiving antennas, whereby 4×8=32 radar channels are available for scanning the field-of-view, when the radar is in a normal operation mode. At least part, for example 3 channels, of the radar channels may be reserved for calibration purposes, whereby the remaining channels, for example 29 channels, may be utilized for monitoring of moving targets by the radar. Accordingly, in this example the multichannel radar of 29 radar channels provides an angular resolution that is enhanced 29/8=3.625 times over a radar having a single transmitting antenna and a receiver array of 8 antennas.

In one application of the radar 104, the radar is used to monitoring targets such as people and/or pets within living facilities. Since the monitoring is based on a radar image rather than video or still images, the monitoring may be performed without compromising privacy of the people and/or the living facilities. This is particularly useful for monitoring in nursing, assisted living and home care applications.

In at least some embodiments, the radar may be connected to one or more processing units 112. The processing unit may be configured to receive at least one of results of scanning radar channels, a radar image generated on the basis of results of the scanning radar channels, information indicating image units in a radar image, and information indicating moving targets within the field-of-view of the radar. Alternatively or additionally, the processing unit may be connected to the radar to control the radar.

In an example a processing unit 112 may comprise a data processor and a memory. The memory may store a computer program comprising executable code for execution by the processing unit. The memory may be a non-transitory computer readable medium. The executable code may comprise a set of computer readable instructions.

In at least some embodiments, the radar and/or processing unit may be connected to a user interface 114 for obtaining input from a user. The input of the user may be used to control the radar and/or the processing unit for monitoring living facilities.

An embodiment concerns an arrangement comprising a multichannel radar 104 and a processor connected to the radar. The arrangement may be sleep monitoring system or a monitoring system for nursing and/or home care. The arrangements may be caused to perform one or more functionalities described herein. Particularly, in nursing and home care it may be of paramount importance to identify situations, where a person is alone in living facilities such that the sleep, sleep apnea or a medical emergency may be detected.

An embodiment concerns an arrangement comprising a multichannel radar 104 and a user interface 114 operatively connected to the radar and a processor connected to the radar to cause: displaying at least one of the radar image, information indicating the number of moving targets, types of the moving targets, information indicating heart rate and information indicating breathing rate. The arrangement provides monitoring of living facilities without compromising privacy. The displayed information may be obtained by performing a method in accordance with at least some embodiments.

An embodiment concerns use of an arrangement comprising a multichannel radar 104 and a user interface 114 operatively connected to the radar and a processor connected to the radar to cause a method according to an embodiment.

It should be appreciated that the user interface may also provide output to the user such. The output may provide that the user may be provided information for example results of scanning radar channels, a radar image generated on the basis of results of the scanning radar channels, information indicating image units in a radar image, and information indicating moving targets within the field-of-view of the radar. In this way the user may monitor operation of the radar and/or processing unit connected to the radar from a remote location.

Examples of the user interfaces comprise devices that may serve for providing output to the user and/or for obtaining input from the user, such as display devices, loudspeakers, buttons, keyboards and touch screens.

In at least some embodiments, the radar and/or processing unit may be connected to an artificial intelligence system 116. The artificial intelligence system may provide adaptation of the monitoring by the radar to the living facilities, where the radar is installed. Examples of the artificial intelligence system comprise computer systems comprising an artificial neural network. The artificial intelligence system may be configured by training the artificial neural network based on user input.

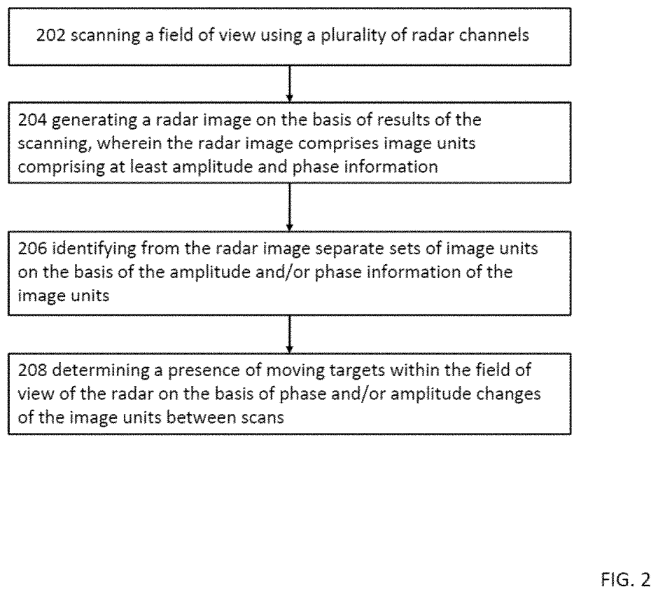

FIG. 2 illustrates an example of a method in accordance with at least some embodiments of the present invention. The method may provide monitoring living facilities. The method may be performed by a multichannel radar or one or more processing units connected to a multichannel radar described with FIG. 1.

Phase 202 comprises scanning, by the multichannel radar or at least one processing unit connected to the radar, a field-of-view within a frequency range of 1 to 1000 GHz, for example between 1 to 30 GHz, 10 to 30 GHz, 30 to 300 GHz or 300 to 1000 GHz using a plurality of radar channels of the radar. Phase 204 comprises generating, by the radar or the processing unit connected to the radar, a radar image on the basis of results of the scanning, wherein the radar image comprises image units comprising at least amplitude and phase information. Phase 206 comprises identifying from the radar image, by the radar or the processing unit connected to the radar, separate sets of image units on the basis of the amplitude and/or phase information of the image units. Phase 208 comprises determining, by the radar or the processing unit connected to the radar, a presence of moving targets within the field-of-view of the radar on the basis of phase and/or amplitude changes of the image units between scans. The movement of the targets is reflected in the amplitude and/or phase of the scans, whereby the targets may be determined as moving targets.

It should be appreciated that the scanning in phase 202 may be performed using signal waveforms transmitted at a carrier frequency selected from a frequency range of 1 to 1000 GHz, for example between 1 to 30 GHz, 10 to 30 GHz, 30 to 300 GHz or 300 to 1000 GHz. However, the frequency range of 30 to 300 GHz may be preferred such that the radar may be configured to have dimensions suitable for indoor installations, while providing the radar to have a sufficient angular resolution.

In an example of determining a presence of moving targets, fluctuations of the phase together with relatively small changes of amplitude of the image units between scans may indicate a micromovement, for example breathing. At the same time image units that surround the image units that have the fluctuations may be substantially constant between scans.

In an example of determining a presence of moving targets, fluctuations of the amplitude of the image units between scans may indicate large movements of the targets, for example a walking person.

In an example of determining a presence of moving targets, periodical changes of the phase together with relatively small changes of the amplitude may indicate micromovements, such as breathing, heart rate, during which the moving target, such as a person may be asleep or at rest.

It should be appreciated a calibration may be performed for determining a presence of moving targets. An initial calibration may be performed by scanning the field-of-view that does not include moving targets. The calibration facilitates determining presence of moving targets, when they enter the field-of-view of the radar. One or more further calibrations may be performed, when it is determined that there are no moving targets in the field-of-view of the radar such that the calibration of the radar may be maintained during the monitoring of the living space.

At least in some embodiments an image unit of a radar image may comprise a range, an azimuth angle, an elevation angle, phase and/or amplitude. The changes of the phase and/or amplitude provide identifying image units to correspond to a moving target. The range and azimuth provide together with the phase and amplitude a 2D radar image. The elevation of the image units provide together with range, azimuth provide, phase and amplitude, a three dimensional (3D) radar image.

An example of phase 202 comprises that the field-of-view of the radar is populated by several antenna beams of the transmitting antennas by using digital Fast Fourier Transform (FFT) beamforming and virtual antenna algorithms. The several antenna beams carry signal waveforms transmitted by the transmitting antennas at a frequency within the frequency range of 1 to 1000 GHz, for example between 1 to 30 GHz, 10 to 30 GHz, 30 to 300 GHz or 300 to 1000 GHz.

An example of phase 204 comprises constructing image units by processing received signals of the radar channels using FFT algorithms and/or correlation algorithms from received signals of the radar channels. One FFT algorithm may be used to derive range, amplitude and phase information from time domain signals received on the radar channels, when the radar is Frequency-modulated continuous-wave radar. When the radar is a coded waveform radar, the correlation algorithms may be used to derive range, amplitude and phase information from time domain signals received on the radar channels. One or more further FFT algorithms may be used for retrieving azimuth and/or elevation angles.

An example of phase 206 comprises processing the radar image by one or more peak search algorithms. Radar images generated based in different scans may be processed to identify separate sets of image units in each radar image for determining phase and/or amplitude changes for determining presence of moving targets in phase 208. It should be appreciated scanning may be performed at a suitable scanning interval to identify separate sets of image units from radar images. Life signs like hear rate and breathing can be further separated by determining and following their change patterns. Further, pets and humans or children and adults, or individuals, can be separated by artificial intelligence or by wearing identification tags that modulate the reflected radar signal or send their own signal.

An example of phase 208 comprises observing the amplitude and/or phase of the target over a time interval. The target may correspond to a separate set of image units identified in phase 206. A single radar image may be considered a snapshot in time, whereby observing image units of the targets over more than one radar images may be used to determine that the targets are moving, when the image units are moved in the radar image.

An example of phase 208 comprises that each separate set determined in phase 206 may be considered a target and the target may be determined to be a moving target on the basis of phase and/or amplitude changes of the image units of corresponding to the target between scans.

In an embodiment, the image units of the radar image further comprise range, azimuth angle and/or elevation angle. In this way separating targets from another and detecting movement of the targets may be performed more accurately.

In an embodiment, phase 206 comprises determining image units belonging to separate sets by grouping the image units on the basis of at least one of: range of the image units; azimuth angle of the image units; elevation angle of the image units; and phase and/or amplitude changes between of the image units between the scans.

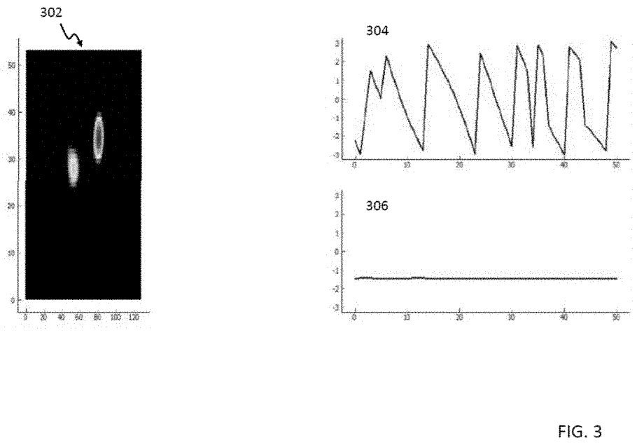

FIG. 3 illustrates an example of a radar image in accordance with at least some embodiments of the present invention. The radar image may be obtained by the method described with FIG. 2. In an example the radar image may be a two dimensional (2D) map of the field-of-view of the radar displayed on a graphical user interface. The radar image may comprise an amplitude plot 302 illustrating amplitude values of image units in the field-of-view of the radar. The radar image may further comprise a phase plot 304, 306 illustrating phase changes between scans. The amplitude plot comprises two separate sets of image units. The sets may be determined on the basis of areas around one or more image units having peak values for amplitude. The phase plot may comprise one phase plot 304 for the set of image units on the left side of the of the amplitude plot. The phase plot may further comprise another phase plot 306 for the set of image units on the right side of the of the amplitude plot. It should be appreciated that each moving target that is detected may be represented by a corresponding phase plot for easy monitoring of the targets. The image units on the left side of the of the amplitude plot may be determined to comprise image units corresponding to a moving target on the basis of phase changes of the phase plot 304. For example, the phase changes between consecutive scans may be determined to exceed a threshold value for determining the image units to comprise image units corresponding to a moving target. On the other hand, the image units on the right side of the of the amplitude plot may be determined not to comprise image units corresponding to a moving target on the basis of phase changes of the phase plot 306. For example, the phase changes between consecutive scans may be determined to be less than the threshold value for determining the image units to comprise image units corresponding to a moving target. Accordingly, in the illustrated example, the number of moving targets may be determined to be one.

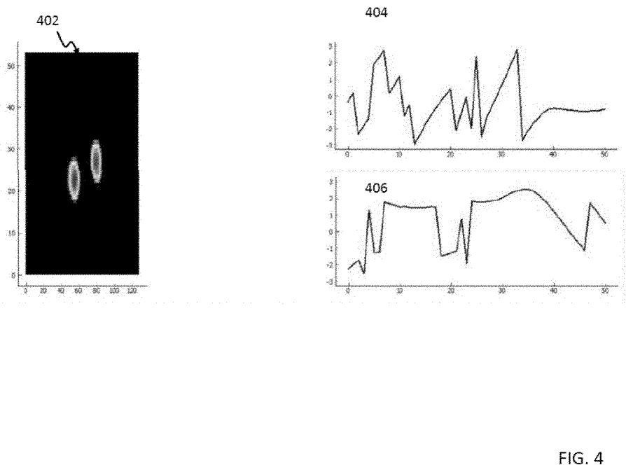

FIG. 4 illustrates an example of a radar image in accordance with at least some embodiments of the present invention. The radar image may be obtained by the method described with FIG. 2. In an example the radar image may be a two dimensional (2D) map of the field-of-view of the radar displayed on a graphical user interface. The radar image may comprise an amplitude plot 402 illustrating amplitude values of image units in the field-of-view of the radar. The radar image may further comprise a phase plot 404, 406 illustrating phase changes between scans. The amplitude plot comprises two separate sets of image units. The sets may be determined on the basis of areas around one or more image units having peak values for amplitude. The phase plot may comprise one phase plot 404 for the set of image units on the left side of the of the amplitude plot. The phase plot may comprise another phase plot 406 for the set of image units on the right side of the of the amplitude plot. It should be appreciated that each moving target that is detected may be represented by a corresponding phase plot for easy monitoring of the targets. The image units on the left and right side of the of the amplitude plot may be determined to comprise image units corresponding to moving targets on the basis of phase changes of the phase plots 404, 406. For example, the phase changes between consecutive scans may be determined to exceed a threshold value for determining the image units to comprise image units corresponding to a moving target. Accordingly, in the illustrated example, the number of moving targets may be determined to be two.

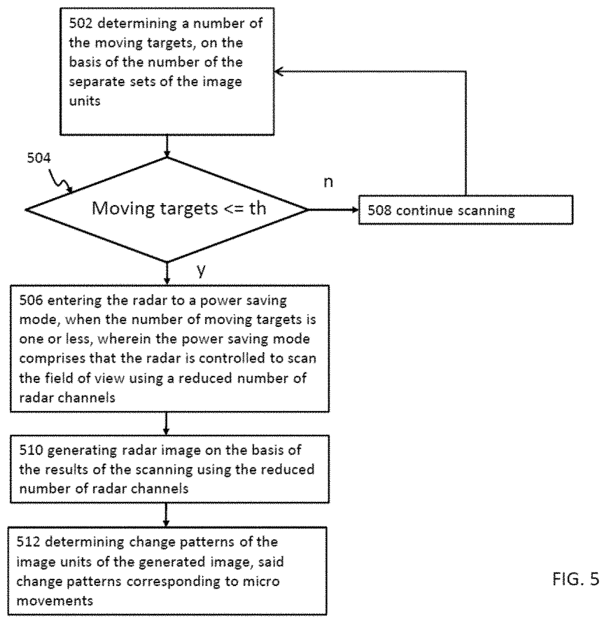

FIG. 5 illustrates an example of a method for controlling a multichannel radar in accordance with at least some embodiments of the present invention. The method may provide power saving in monitoring living facilities by the multichannel radar. The method may be performed by the multichannel radar or one or more processing units connected to the multichannel radar described with FIG. 1, when a radar image has been generated by scanning a field-of-view of the radar and a presence of one or more moving targets has been determined in accordance with the method of FIG. 2.

Phase 502 comprises determining a number of the moving targets, on the basis of the number of the separate sets of the image units. Phase 504 comprises determining whether the number of the moving targets is less than equal to a threshold value, for example an integer value such as one. Phase 506 comprises entering the radar to a power saving mode, when the number of moving targets is less than equal to the threshold value, wherein the power saving mode comprises that the radar is controlled to scan the field-of-view using a reduced number of radar channels, for example one radar channel. Accordingly, in the power saving mode only one radar channel may be active and the other radar channels may be passive. In this way, the field-of-view may be scanned with a shorter time period between consecutive scans than when a higher number of radar channels, e.g. all radar channels or substantially all radar channels, were used for scanning. The shorter time period between the scans provides that micro movements of the target within the field-of-view may be monitored by the radar more accurately. A micro movement may be a movement of a part of the target, for example a movement of the chest by respiration and a movement of the chest by heartbeat.

In an example of phase 502, each separate set may be considered a target and the target may be determined to be a moving target on the basis of phase and/or amplitude changes of the image units of corresponding to the target between scans, in accordance with phase 208 of FIG. 2.

On the other hand, when it is determined that the number of moving targets is not less than equal to the threshold value, phase 508 is performed, where scanning the field-of-view of the radar is continued by performing one or more scans by a number of radar channels that is sufficient for generating a radar image for determining presence of multiple moving targets within the living facilities, for example in a normal operation mode of the radar. After one or more scans have been performed in phase 508, the phase 502 may be performed anew.

In an embodiment, in the power saving mode change patterns of the image units corresponding to micro movements such as at least one of heart rate and breathing are determined. In this way the condition of the monitored target such as breathing and/or heart rate may be followed more accurately. The change patterns may be determined by phases 510 and 512. Phase 510 comprises generating a radar image on the basis of the results of the scanning using the reduced number of radar channels in the power saving mode. Phase 512 comprises determining change patterns of the image units of the generated image, said change patterns corresponding to micro movements such as at least one of heart rate and breathing. The change patterns of the micro movements such as heart rate and breathing may be used to determine information indicating a rate, e.g. heart rate and/or breathing rate which may be displayed on a user interface.

In an embodiment, the radar is triggered to leave the power saving mode after a time interval has passed and/or on the basis of a trigger signal. In this way the phases 502 and 504 may be performed anew such that detecting a change in the presence of moving targets may be facilitated. When the power saving mode is left, the radar may be caused to enter another operation mode, for example the operation mode of the radar prior to entering the power saving mode, such as a normal operation mode.

In an example the radar is triggered after 1 to 10 s time period in the power saving mode to leave the power saving mode. The power saving mode may be returned by performing the phases 502, 504 and 506, after which the radar may be triggered to leave the power saving mode again. In another example the radar is triggered to leave the power saving mode by a trigger signal. The trigger signal may be information derived from a radar image, such as image units. Examples of the trigger signal comprise a rate of micro movements such as a heart rate and breathing rate. The rate of micro movement may be evaluated against a threshold to determine the rate as a trigger signal. For example a heart rate or breathing rate exceeding a threshold or less than a threshold may be used for a trigger signal.

Further examples of triggers for the radar to leave the power saving mode comprise, when the measurements indicate that a person gets up from bed, when more than one people are detected in the field-of-view, when data obtained by the measurements is unclear.

It should be appreciated that after the power saving mode has been entered in phase 506, the power saving mode may be changed to another operation mode, for example to a normal operation mode, where a higher number of radar channels, for example substantially all radar channels, are used for scanning. The operation mode may be changed, for example when a time interval has been elapsed. Said another operation mode may be the operation mode of the radar that preceded the radar entering the power saving mode. When the radar is not in the power saving mode, the power saving mode may be again entered in accordance with phases 502 and 504.

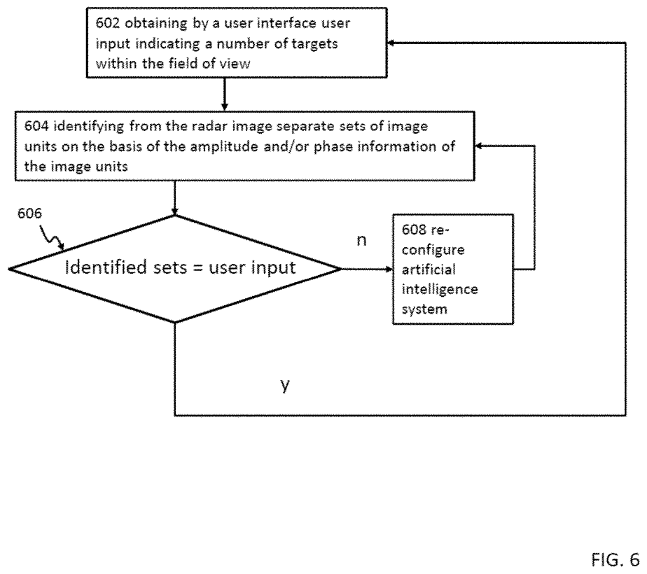

FIG. 6 illustrates identifying image units corresponding to targets by an artificial intelligence system in accordance with at least some embodiments of the present invention. The method may be performed by a multichannel radar or one or more processing units connected to a multichannel radar that are connected to an artificial intelligence system and a user interface described with FIG. 1. The artificial intelligence system may have an initial configuration that provides at least identifying from a radar image separate sets of image units on the basis of the amplitude and/or phase information of the image units. It should be appreciated that in addition to identifying from a radar image separate sets of image units, the artificial intelligence system may be in principle used to detect any occurrence of previously undetected patterns, e.g. “fingerprints”. Also other information of the image units such as range, azimuth angle, elevation angle, and phase and/or amplitude changes between of the image units between the scans may be used by the artificial intelligence system for the identifying. The initial configuration may be received by user input or the initial configuration may be predefined to a configuration of the artificial intelligence system. The method may provide that monitoring is adapted to the living facilities, where the radar is installed. The method may be performed, when a radar image has been generated by scanning a field-of-view of the radar in accordance with the method of FIG. 2, for example during a training phase of the artificial intelligence system. After the training phase is complete, the artificial intelligence system is configured to support the monitoring of the living facilities by the radar by identifying a number of targets within a radar image.

Phase 602 comprises obtaining by the user interface user input indicating a number of targets within the field-of-view. Phases 604 and 606 provide determining by the artificial intelligence system a correspondence between separate sets of image units of the radar image and the number of targets within the field-of-view indicated by the user input. Phase 604 comprises identifying, by the artificial intelligence system, from the radar image separate sets of image units on the basis of the amplitude and/or phase information of the image units, in accordance with phase 206 of FIG. 2. Phase 606 comprises determining whether a number of the separate sets identified in Phase 604 correspond with the number of targets within the field-of-view indicated by the user input. Phase 606 may provide data indicating a result of determining the correspondence. The data may be utilized in teaching the artificial intelligence system in a supervised learning method.

When the correspondence is determined, thus the result of phase 606 is positive, the artificial intelligence system is capable, using its current configuration, of identifying separate sets of image units corresponding to targets, and the method proceeds from phase 606 to phase 602 to obtain further input from the user and to identify sets of image units from a new radar image in phase 604. When the correspondence is not determined, thus the result of phase 606 is negative, the method proceeds from phase 606 to phase 608 to re-configure the artificial intelligence system and to phase 604, where the artificial intelligence system is used to perform identification of the separate sets using the new configuration determined in phase 608. In this way the new configuration of the artificial intelligence system may provide in phase 604 a new result that may be evaluated against the user input in phase 606. In this way, a configuration of the artificial intelligence system may be determined that provides identifying of separate sets corresponding to targets in the field-of-view.

It should be appreciated that the phases 602, 604, 606 and 608 may be repeated until the correspondence between separate sets of image units of radar images and the number of targets within the field-of-view indicated by the user input is obtained with sufficient certainty. In an example, the sufficient certainty may be determined based on a relationship of positive results and negative results determined in phase 606, when multiple radar images are processed by the phases 602 to 608. When the relationship is 99% of positive results it may be determined that the configuration of the artificial intelligence system has been adapted for monitoring the living facilities, where the radar is installed and the artificial intelligence system is configured to support the monitoring of the living facilities by the radar. After the sufficient certainty has been achieved the artificial intelligence system may identify image units corresponding to targets from the radar image, for example in phase 206.

At least some embodiments comprise a plurality of types of moving targets. Examples of the types comprise pets, humans, children and/or adults, and a type of target is defined by one or more patterns, and the separate sets of the image units are compared to the types of targets for identifying the separate sets to one or more of the types of the moving targets.

An embodiment concerns a method for identifying image units corresponding to a specific type of targets by an artificial intelligence system. Accordingly, the artificial intelligence system may be configured to support monitoring of the living facilities by a multichannel radar by identifying a number of targets of the specific type within a radar image. Types of the targets may comprise pets, humans, children and/or adults. The method may be performed in accordance with the method described with FIG. 6 with the difference that phase 602 comprises obtaining by the user interface user input indicating a number of targets of the specific type within the field-of-view. Accordingly, the method may applied for identifying image units corresponding to any of the types based on obtaining input from the user indicating the number of the specific type of targets. One type of targets should be selected for the method at time to facilitate obtaining a configuration of the artificial intelligence system capable of identifying separate sets of image units corresponding to targets of the specific type.

An embodiment comprises a non-transitory computer readable medium having stored thereon a set of computer readable instructions that, when executed by a multichannel radar or at least one processor connected to a multichannel radar, cause the multichannel radar or the one processor and the multichannel radar to at least: scanning a field-of-view within a frequency range from 1 to 1000 GHz, for example between 1 to 30 GHz, 10 to 30 GHz, 30 to 300 GHz or 300 to 1000 GHz, using a plurality of radar channels of the radar; generating a radar image on the basis of results of the scanning, wherein the radar image comprises image units comprising at least amplitude and phase information; identifying from the radar image separate sets of image units on the basis of the amplitude and/or phase information of the image units; and determining a presence of moving targets within the field-of-view of the radar on the basis of phase and/or amplitude changes of the image units between scans.

An embodiment comprises a computer program configured to cause a method in accordance with at least some embodiments described herein. The computer program may comprise executable code that may be executed by a processing unit for causing the embodiments.

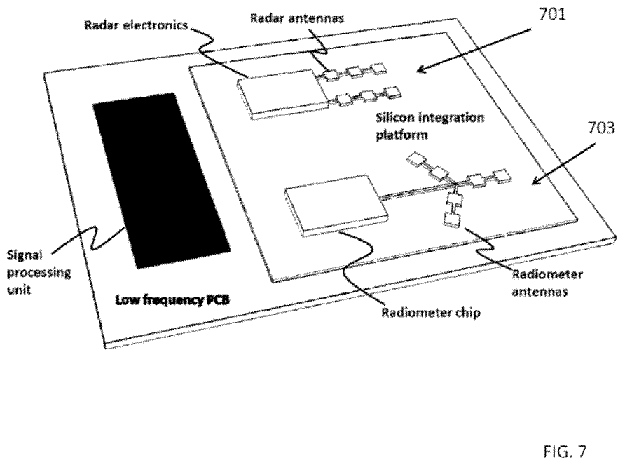

FIG. 7 illustrates an example of an arrangement in accordance with at least some embodiments of the present invention. The arrangement comprises a multichannel radar 701 and a microwave imaging radiometer 705. The multichannel radar may be the multichannel radar described with FIG. 1. The microwave imaging radiometer measures energy of thermal electromagnetic radiation at millimetre-to-centimetre wavelengths (frequencies of 1-1000 GHz) known as microwaves. The arrangement may be configured to cause a method in accordance with an embodiment.

In an embodiment, the arrangement may comprise one or more processors 710 that are connected to the multichannel radar and the microwave imaging radiometer. The processor may be data processor of a processing unit comprising the data processor and a memory. The arrangement may comprise and at least one memory including computer program code. The at least one memory and computer program code may be configured to, with the processors, to cause the performance of the arrangement. In an example arrangement may have a single processor connected to both to the radar 701 and the microwave imaging radiometer 705.

Examples of the processors 710 comprise single core and multicore processors. A processor may be a signal processor adapted to process radar signals and/or microwave imaging radiometer signals.

In an example, the multichannel radar 701 may comprise radar electronics 702 and radar antennas 704 controlled by the radar electronics. The microwave imaging radiometer 705 may comprise a radiometer chip 706 and radiometer antennas 708 controlled by the radiometer chip. On the other hand the

It should be appreciated that the microwave imaging radiometer 705 and the multichannel radar 701 may be aligned such that their field-of-views are at least partly overlapping. Therefore, it can be understood that a mapping may be provided between the field-of-view of the microwave radiometer and the multichannel radar such that the microwave radiometer and the multichannel radar may be have a single field-of-view.

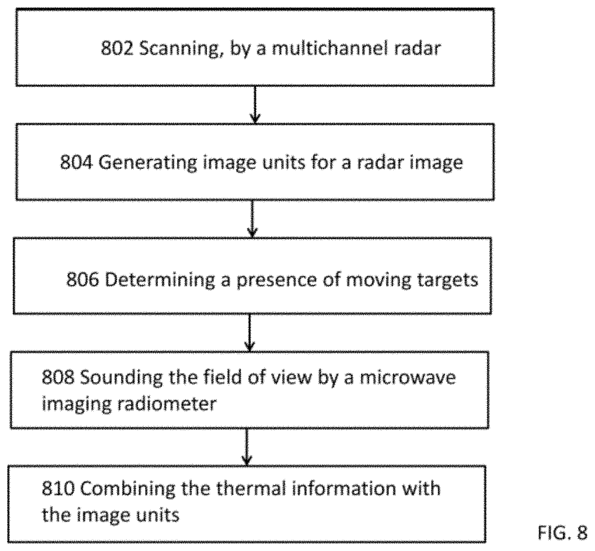

FIG. 8 illustrates an example of a method in accordance with at least some embodiments of the present invention. The method provides image units for vital sign monitoring based on movement and thermal information in the field-of-view of an arrangement comprising a multichannel radar and a microwave radiometer. The arrangement may be installed to living facilities for monitoring moving targets, e.g. persons, within the living facilities. The method may be performed by the arrangement described with FIG. 7. Examples of the living signs comprise breathing rate, heart rate and body temperature.

Phase 802 comprises scanning, by a multichannel radar or at least one processing unit connected to the radar, a field-of-view within a frequency range from 1 to 1000 GHz, for example between 1 to 30 GHz, 10 to 30 GHz, 30 to 300 GHz or 300 to 1000 GHz, using a plurality of radar channels of the radar. In an example phase 802 may be performed in accordance with phase 202 of FIG. 2.

Phase 804 comprises generating, by the radar or the processing unit connected to the radar, image units for a radar image on the basis of results of the scanning, wherein the image units comprise at least amplitude and phase information. In an example phase 802 may be performed in accordance with phase 204 of FIG. 2.

Phase 806 comprises determining, by the radar or the processing unit connected to the radar, a presence of moving targets within the field-of-view of the radar on the basis of phase and/or amplitude changes of the image units between scans. In an example phase 802 may be performed in accordance with phases 206 and 208 of FIG. 2. In an example image units associated with a person may be the separate sets of image units determined in phase 206 that are determined as moving targets in accordance with phase 208.

Phase 808 comprises sounding the field-of-view by a microwave imaging radiometer for obtaining thermal information associated with the field-of-view. The thermal information may comprise energy of thermal electromagnetic radiation.

Phase 810 comprises combining the thermal information or information determined based on the thermal information with the image units of the moving targets. In this way the image units may comprise amplitude, phase and thermal information, which facilitate monitoring the living signs.

In an example, phase 810 comprises synchronizing and correlating the results of the scanning with the thermal information obtained by the sounding. The synchronized and correlated data comprising amplitude, phase and thermal information may be stored to the image units.

In an alternative embodiment, phase 810 comprises determining a temperature representative of a body temperature of at least one of the moving targets based on the thermal information obtained by the microwave imaging radiometer. In this way the determined body temperature may be stored to the image units.

In an alternative embodiment, phase 810 comprises that a body temperature or the thermal information may be selectively combined with the image units of the moving targets. In this way, possible errors and inaccuracies that affect the thermal information obtained by the microwave imaging radiometer may be compensated. In an example, when the thermal information associated with the image units is compensated, use of the temperature or thermal information may be prevented or ignored. Then, a temperature or thermal information previously combined with the image units, e.g. by execution of the phases 802 to 810, may be maintained without updating the temperature or thermal information of the image units with later temperature or thermal information obtained by scanning and sounding performed later in accordance with phases 802 and 808.

In an example phase 810 comprises that the thermal information or information determined based on the thermal information combined to the image units may form an image for representing living signs of moving targets. In this way the thermal information may be used to monitor the moving targets, e.g. persons, and their medical condition. The image may be displayed on a user interface connected to the arrangement described in an embodiment.

In an embodiment, a method may comprise, for example in connection with phase 806, determining image units associated with a person in the field-of-view, and determining the image units associated with the person to indicate a sign of life, a temperature, heart rate and/or a breathing rate. In this way the living signs of the persons in the field-of-view may be monitored.

In an embodiment, a method may comprise, for example in connection with phase 806, that a breathing and/or heart rate are determined based on periodical changes of phase together with relatively small changes of amplitude of an image unit.

In an embodiment phase 810 comprises determining a medical condition of the person based on a change pattern of the image units. The image units may comprise amplitude, phase information and the thermal information or information determined based on the thermal information. Then, absolute values and changes of the amplitude, phase information and the thermal information or information determined based on the thermal information may be compared with one or more patterns corresponding to medical conditions. A medical condition may be determined, when a match between the image units and at least one of the patterns is determined. It should be appreciated that a medical condition may be defined by a combination of patterns, whereby a match between the image units and all the patterns may be needed for determining the medical condition.

In an embodiment phase 810 comprises determining vital signs and/or a medical condition of the person based on the thermal information or information determined based on the thermal information combined with the image units. In this way the thermal information in combination with the information of movement in the field-of-view may be used for determining the vital signs and/or the medical condition.

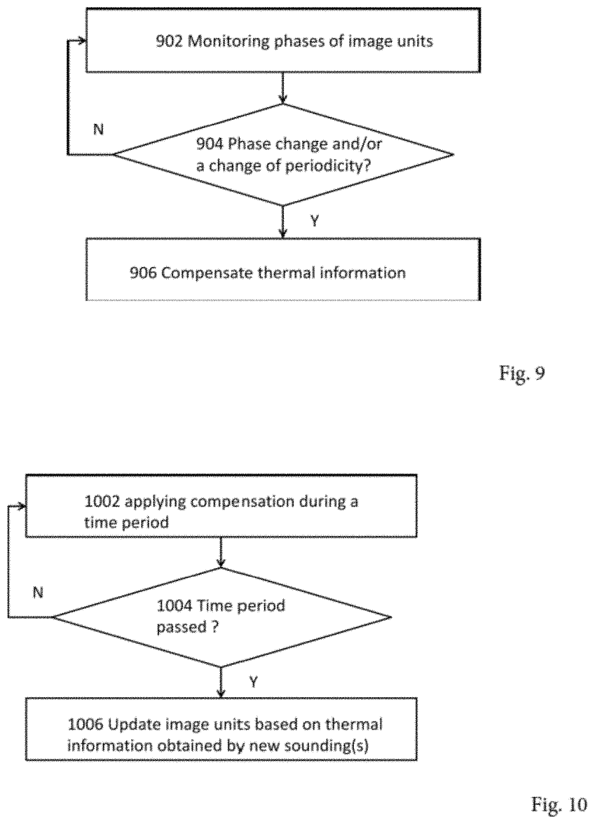

FIG. 9 illustrates an example of a method in accordance with at least some embodiments of the present invention. The method may be performed by the arrangement described with FIG. 7.

Phase 902 comprises monitoring phases of image units of one or more moving targets. The monitoring provides image units from a field-of-view of the multichannel radar and a microwave imaging radiometer. The image units may comprise at least amplitude and phase information combined with thermal information or a temperature representative of a body temperature determined based on the thermal information. In an example phase 902 may comprise repeating the method of FIG. 8, at least phases 802 and 804, continuously such that the image units may be kept up-to-date.

Phase 904 comprises determining if the image units indicate large movements based on a phase change of the image units and/or a change of periodicity of the image units. If the image units indicate large movements, the method proceeds to phase 906. If not, the method may proceed to phase 902.

In an example, in phase 904, the large movements may be determined based on fluctuations of the amplitude and/or phase of the image units between scans. For example, a large movement may be determined, if a change of the amplitude and/or phase of the image units is large and abrupt. When the fluctuations of the amplitude and/or phase are aperiodic, it may be further determined that the movement is not a periodic movement such as a breathing rate or a heart rate.

In an example, phase 904 comprises that a phase change of the image units and/or a change of periodicity of the image units may be determined based on comparing amplitude and/or phase of the image units to corresponding threshold values for amplitude and phase.

In an example, phase 904 comprises that the phase change of the image units and/or a change of periodicity of the image units may be determined by an artificial intelligence system connected to the arrangement or the radar.

Phase 906 comprises, compensating the thermal information or information determined based on the thermal information associated with the image units. In this way, effect to the image units caused by possible errors and inaccuracies in the thermal information obtained by the microwave imaging radiometer may be reduced.

Examples of compensating the thermal information comprise preventing use of thermal information, ignoring thermal information and applying a correction factor to thermal information.

FIG. 10 illustrates an example of a method in accordance with at least some embodiments of the present invention. The method provides compensating thermal information associated with image units. The method may be performed in phase 906 of FIG. 9 for example. Phase 1002 comprises applying compensation during a time period. In an example use of the thermal information is prevented or ignored in association with the image units for the duration of the time period. The thermal information or information determined based on the thermal information may be obtained based on the sounding performed in phase 808 of FIG. 8. The sounding may be performed a plurality of times, whereby the information obtained by the sounding comprises the newest information and a history of the information obtained in one or more previous time instants. Then, if 1004 the time period has not passed, compensation may be applied in accordance with phase 1002 such that information obtained by the sounding performed e.g. in phase 808 during the time period may be prevented or ignored for reducing effect to the image units caused by possible errors and inaccuracies in the thermal information. During the time period that the compensation is applied, the image units comprise the thermal information or information determined based on the thermal information obtained in one or more time previous time instants.

On the other hand, if 1004 the time period has passed, the method may proceed to phase 1006, where image units may be updated with thermal information or information determined based on the thermal information obtained by at least one sounding performed after the time period has passed. Accordingly, the image units may be combined with thermal information or information determined based on the thermal information in accordance with phases 808 and 810.

The time period during which the use of the thermal information is prevented or ignored in phase 1002 may be a pre-set time period. On the other hand the time period may be determined adaptively based on a temperature difference. The temperature difference may be a difference between body temperatures determined based on the temperature information obtained by at least one previous sounding by the microwave imaging radiometer and at least one later sounding by the microwave imaging radiometer. In this way, the time period may be determined adaptively based on the temperature difference. Alternatively or additionally, also the context, where the microwave imaging radiometer is used for sounding may be used to determine the time period. In an example, if the temperature difference is above a threshold value for temperature, the time period may be higher than if the temperature difference is below the threshold value. The temperature prevailing in the living facilities may be determined based on temperature information obtained for image units that are not moving targets. Alternatively or additionally a separate temperature sensor may be connected to the arrangement to provide the temperature prevailing in the living facilities.

FIG. 11 illustrates an example of antenna array layout of a microwave imaging radiometer in accordance with at least some embodiments of the present invention. The microwave imaging radiometer may be the microwave imaging radiometer illustrated in FIG. 7. On the other hand the microwave imaging radiometer may be a microwave imaging radiometer in an arrangement, where the microwave imaging radiometer has a common pre-amplifier and antenna with a multichannel radar.

The microwave imaging radio meter may comprise an antenna that is an antenna array 1102. A layout of the antenna array may comprise antenna elements 1104, where the antenna elements are configured into a shape comprising three arms 1106. FIG. 11 illustrates the array in a two-dimensional plane. Center of the arms may be formed by three antenna elements corresponding to each of the arms. The array formed by the arms may have the shape of letter Y or a propeller. Accordingly, the array may be referred to e.g. a Y-array.

FIG. 12 illustrates an example of a receiver 1202 for an antenna element of a microwave imaging radiometer in accordance with at least some embodiments of the present invention. The receiver may be a receiver of an antenna element in an antenna array layout of FIG. 11 for example. Each of the antenna elements of the microwave imaging radiometer may have their own receiver.

The receiver may comprise one or more components for forming an intermediate frequency signal of a radio signal received by an antenna. The receiver may comprise a correlator 1204 for cross-correlating intermediate frequency signals of intermediate frequency signals formed by receivers of one or more other antenna elements of the microwave imaging radiometer. It should be appreciated that the receiver may further comprise a digitizer for outputting a digital signal, a low-noise-amplifier (LNA) a band-pass filter (BPF), a quadrature downconverter, a buffer amplifier, a phase shifter and a signal generator (LO) that may be connected for processing the radio signal received from the antenna.

FIG. 13 illustrates an example of a method for sounding a field-of-view by a microwave imaging radiometer in accordance with at least some embodiments of the present invention. The sounding provides thermal information associated with the field-of-view of the microwave imaging radiometer. The sounding may be performed in phase 808 of FIG. 8, for example.

Phase 1302 comprises measuring a field-of-view of the microwave imaging radiometer. All antenna elements of the microwave imaging radiometer may measure the same field-of-view.

Phase 1304 comprises cross-correlating intermediate frequency signals of receivers of all the antenna elements against each other.

Phase 1306 comprises forming an interferometer by each correlated antenna pair. The interferometer is capable of measuring a particualt spatial harmonic of the brightness temperature.

Phase 1306 comprises generating, e.g. reconstructing or synthesizing, a two-dimensional image of the FOV by an inverse transform.

An embodiment concerns an arrangement comprising a multichannel radar, and a microwave imaging radiometer, wherein the multichannel radar, and a microwave imaging radiometer have a common pre-amplifier and antenna. In this way, the use of the pre-amplifier and the antenna may be shared between the microwave imaging radiometer and the multichannel radar and the arrangement may effectively operate as microwave imaging radiometer or the multichannel radar at a time. In an example the common pre-amplifier and antenna may be shared based on time, a number of soundings, and/or a number of scannings. In an example, the multichannel radar and the microwave imaging radiometer may be configured to perform 10 measurements per second. Accordingly, the shared use of the pre-amplifier and the antenna may be arranged within a time period of one second such that after the pre-amplifier and the antenna have been used for scanning by the multichannel radar, the pre-amplifier and the antenna may be used for sounding by the microwave imaging radiometer. The antenna layout of the arrangement may be in accordance with the layout illustrated in FIG. 11.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the foregoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### ACRONYMS LIST

2D Two Dimensional

3D Three Dimensional

BPF Band-Pass Filter

FFT Fast Fourier Transform

FOV Field-of-View

LNA Low-Noise-Amplifier

LO Signal Generator

MIMO Multiple Input Multiple Output

MISO Multiple Input Single Output

SIMO Single Input Multiple Output

UWB Ultra-WideBand

### REFERENCE SIGNS LIST

102 Field-of-view

104 Multichannel radar

106 Transmitting antennas

108 Receiving antennas

110 Targets

112 Processing unit

114 User interface

116 Artificial intelligence system

202 to 208 Phases of FIG. 2

302 Amplitude plot

304, 306 Phase plot

402 Amplitude plot

404, 406 Phase plot

502 to 512 Phases of FIG. 5

602 to 608 Phases of FIG. 6

701 Multichannel radar

702 Radar electronics

704 Radar antennas

705 Microwave imaging radiometer

706 Radiometer chip

708 Radiometer antennas

710 Processor

802 to 810 Phases of FIG. 8

902 to 906 Phases of FIG. 9

1002 to 1006 Phases of FIG. 10

1. A method of providing image units for vital sign monitoring, comprising:
scanning, by a multichannel radar or at least one processing unit connected to the radar, a field of view within a frequency range from 1 to 1000 GHz, for example between 1 to 30 GHz, 10 to 30 GHz, or 30 to 300 GHz, using a plurality of radar channels of the radar;
generating, by the radar or the processing unit connected to the radar, image units for a radar image on the basis of results of the scanning, wherein the image units comprise at least amplitude and phase information;
determining, by the radar or the processing unit connected to the radar, a presence of moving targets within the field of view of the radar on the basis of phase and/or amplitude changes of the image units between scans;
sounding the field of view by a microwave imaging radiometer for obtaining a thermal information associated with the field of view; and
combining the thermal information or information determined based on the thermal information with the image units of the moving targets, wherein movement information is used to adaptively process the thermal information.

2. The method according to claim 1, further comprising:
monitoring phases of image units of one or more moving targets and if the image units indicate large movements based on a phase change of the image units and/or a change of periodicity of the image units, the thermal information associated with the image units is compensated.

3. The method according to claim 2, wherein the compensation is applied during a time period.

4. The method according to claim 3, further comprising updating the image units with thermal information or information determined based on the thermal information obtained by at least one sounding performed after the time period has passed.

5. The method according to claim 1, further comprising:
determining a temperature representative of a body temperature of at least one of the moving targets based on the thermal information obtained by the microwave imaging radiometer.

6. The method according to claim 1, further comprising:
determining image units associated with a person in the field of view; and
determining the image units associated with the person to indicate a sign of life, a temperature, heart rate and/or a breathing rate.

7. The method according to claim 6, wherein the breathing and/or heart rate are determined based on periodical changes of phase together with relatively small changes of amplitude of an image unit.

8. The method according to claim 1, further comprising:
determining a medical condition of a person based on a change pattern of the image units.

9. The method according to claim 1, further comprising:
determining the vital signs and/or a medical condition of a person based on the thermal information or information determined based on the thermal information combined with the image units.

10. An arrangement comprising:
multichannel radar, and
microwave imaging radiometer, wherein arrangement is configured to cause:
scanning, by the multichannel radar or at least one processing unit connected to the radar, a field of view within a frequency range from 1 to 1000 GHz, for example between 1 to 30 GHz, 10 to 30 GHz, or 30 to 300 GHz or 300 to 1000 GHz, using a plurality of radar channels of the radar;
generating, by the radar or the processing unit connected to the radar, image units for a radar image on the basis of results of the scanning, wherein the image units comprise at least amplitude and phase information;
determining, by the radar or the processing unit connected to the radar, a presence of moving targets within the field of view of the radar on the basis of phase and/or amplitude changes of the image units between scans;
sounding the field of view by the microwave imaging radiometer for obtaining a thermal information associated with the field of view; and
combining the thermal information obtained by the microwave imaging radiometer with the image units of the moving targets, wherein movement information is used to adaptively process the thermal information.

11. The arrangement according to claim 10, further comprising: at least one processor; and at least one memory including computer program code, the at least one memory and computer program code configured to, with the at least one processor, cause the performance of the arrangement.

12. The arrangement according to claim 10, wherein the microwave imaging radiometer has a common pre-amplifier and antenna with the multichannel radar.

13. A non-transitory computer readable medium having stored thereon a set of computer readable instructions that, when executed by at least one processor, cause an arrangement to at least:
scanning, by a multichannel radar or at least one processing unit connected to the radar, a field of view within a frequency range from 10 to 30 GHz, or 30 to 300 GHz or 300 to 1000 GHz, using a plurality of radar channels of the radar;
generating, by the radar or the processing unit connected to the radar, image units for a radar image on the basis of results of the scanning, wherein the image units comprise at least amplitude and phase information;
determining, by the radar or the processing unit connected to the radar, a presence of moving targets within the field of view of the radar on the basis of phase and/or amplitude changes of the image units between scans;
sounding the field of view by a microwave imaging radiometer for obtaining a thermal information associated with the field of view; and
combining the thermal information obtained by the microwave imaging radiometer with the image units of the moving targets, wherein movement information is used to adaptively process the thermal information.

14. (canceled)
